(19) United States
(12) Patent Application Publication
(10) Pub. No.: US 2017/0002024 A1
(43) Pub. Date: Jan. 5, 2017

(54) BENZOTHIADIAZOLE-BASED CONJUGATED MOLECULES CAPABLE OF FORMING FILMS ON CONDUCTIVE SURFACES BY ELECTROCHEMICAL METHOD

(71) Applicant: HITACHI CHEMICAL COMPANY AMERICA, LTD. & HITACHI CHEMICAL COMPANY, LTD., San Jose, CA (US)

(72) Inventors: ANANDO DEVADOSS, IRVINE, CA (US); CUIHUA XUE, IRVINE, CA (US); HAN-KUAN ANTHONY TSAI, IRVINE, CA (US)

(21) Appl. No.: 15/125,913

(22) PCT Filed: Mar. 13, 2015
(86) PCT No.: PCT/US15/20403

§ 371 Date: Sep. 13, 2016

Related U.S. Application Data
(60) Provisional application No. 61/953,444, filed on Mar. 14, 2014.

(51) Int. Cl. C07D 513/04; C09D 165/00; C25D 5/54; C25D 9/02; C25D 17/10; C25D 5/48; C08G 61/12; C09D 5/24

(52) U.S. Cl. CPC C07D 513/04; C08G 61/126; C09D 165/00; C09D 5/24; C25D 9/02; C25D 17/10; C25D 5/48; C25D 5/54; C08G 2261/77; C08G 2261/3246; C08G 2261/3241; C08G 2261/3223; C08G 2261/148; C08G 2261/1426; C08G 2261/145

(57) ABSTRACT

The present disclosure provides new materials that combine the advantages of well-defined polymeric starting materials and the convenience of surface modification by physical methods into one package and, thus, offers a general and powerful platform suitable for use in numerous applications.

### FIELD

The present disclosure relates to monomeric and polymeric compositions, uses and related methods.

### BACKGROUND

In this specification where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

Surface modification of monomeric and polymeric compositions has been the subject of intense interest for its application in many areas, such as adhesion, printing on films, dyeing of fabrics, oil repellency in air, food packaging, cell culture dishes, cell supports in fermentation processes, biodegradable polymers, biosensors and diagnostic assays, sterile packaging, protein and cell separations, and the like.

Thus, there is a need for modifying the surface of monomeric and polymeric compositions.

While certain aspects of conventional technologies have been discussed to facilitate disclosure of the invention, Applicants in no way disclaim these technical aspects, and it is contemplated that the claimed invention may encompass or include one or more of the conventional technical aspects discussed herein.

### SUMMARY

According to one aspect of the invention, the present invention provides one or more molecules having a formula (1), (2), (3), (4), (5), or (6);
wherein:

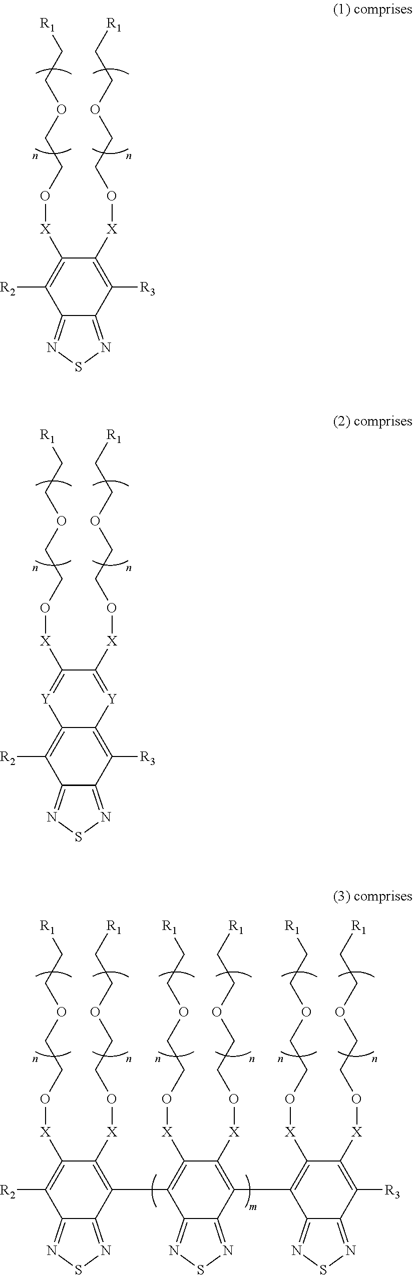

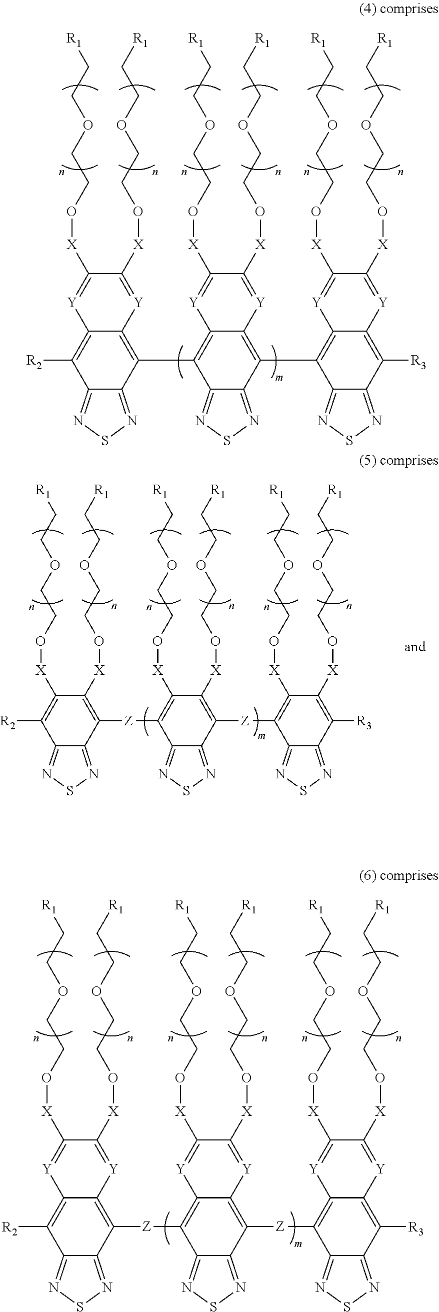

wherein
R₁=OH, COOH, NH₂, SO₃H, SR₄COOH, SR₄SO₃H, maleimide, N-hydroxysuccinimide, a protein, a nucleic acid, or a nanoparticle;
R₂=H, an alkene, an aromatic monomer, an aromatic oligomer, a heterocyclic monomer, or a heterocyclic oligimer;
R₃=R₂, a ligand, a metal, a protein, a nucleic acid, or a nanoparticle;
R₄=—(CH₂)ₜ—, —(CH₂—O—CH₂)ₜ—, an alkene, a polyalkene, an aromatic monomer, an aromatic polymer, a heterocyclic monomer, or a heterocyclic polymer;
X=N, S, O, P, —(CH₂)ₜ—, an aromatic monomer, or a heterocyclic monomer;
Y=N, S, O, or P;
Z=—(CH₂)ₜ—, an alkene, a polyalkene, an aromatic monomer, an aromatic polymer, a heterocyclic monomer, or a heterocyclic polymer;
m is 1 to 20;
n is 1 to 20; and
t is 1 to 20.

According to another aspect, the present invention provides one or more molecules having structural formula (7), (8), (9), or (10),
wherein:

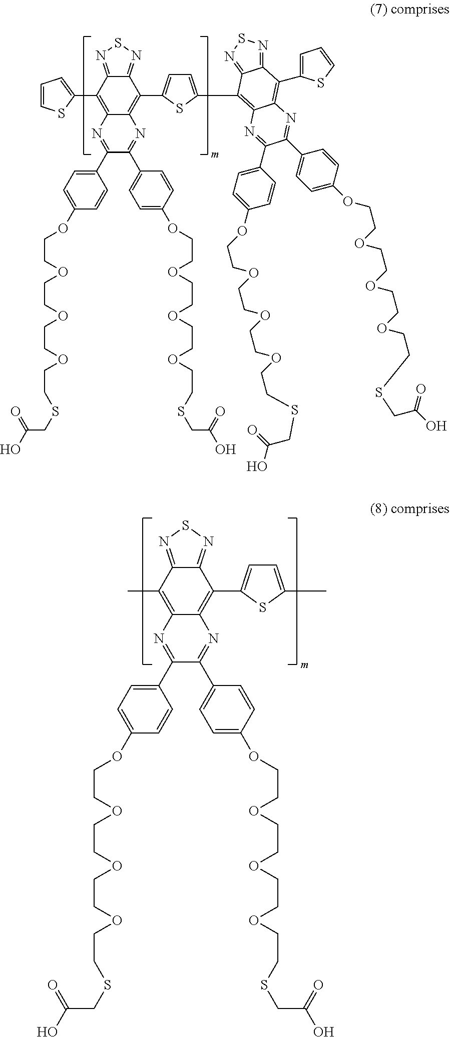

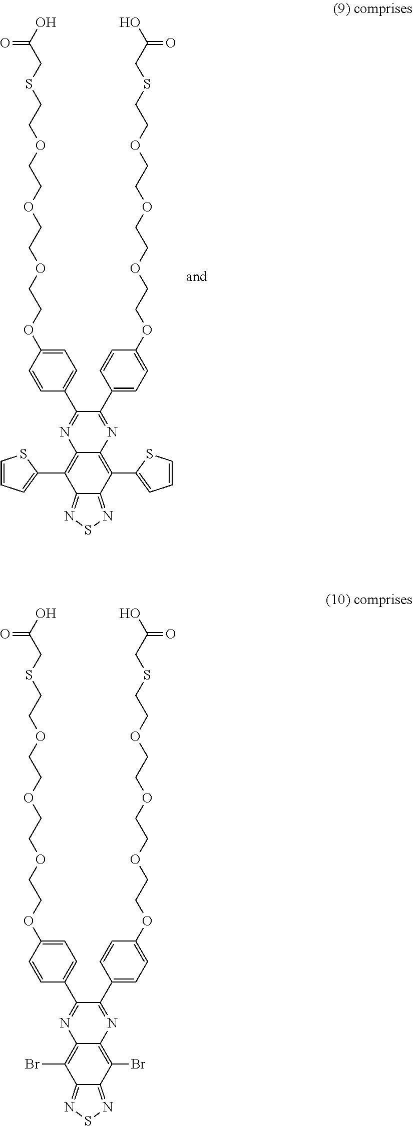 [SMILES: O=C(O)CSCCOCCOCCOCCOc1ccc(-c2nc3c(-c4cccs4)c4nsnc4c(-c4cccs4)c3nc2-c2ccc(OCCOCCOCCOCCSCC(=O)O)cc2)cc1 ; O=C(O)CSCCOCCOCCOCCOc1ccc(-c2nc3c(Br)c4nsnc4c(Br)c3nc2-c2ccc(OCCOCCOCCOCCSCC(=O)O)cc2)cc1]

wherein molecules (7), (8), (9), and (10) are specific embodiments of molecules (1), (2), (3), (4), (5), or (6). More specifically, molecules (7) and (8) are specific embodiments of molecule (3), molecule (9) is a specific embodiment of molecule (1), and molecule (10) is a specific embodiment of molecule (2).

According to an additional aspect, the present invention provides one or more compound comprising one or more molecules (1) to (10).

According to yet another aspect, the present invention provides a substrate or surface modified by one or more molecules (1) to (10).

According to still another aspect, the present invention provides a method. Methods performed according to the principles of the present invention may generally comprise one or more of the following steps, which may or may not be performed in the order below:
optionally, preparing the surface of the electrode to be modified by one or more molecules (1) to (10);
preparing a solution comprising one or more molecules (1) to (10);
placing the solution into communication with the surface of the electrode to be modified;
applying a predetermined potential between the surface of the electrode to be modified and at least one counter electrode, for a predetermined period of time;
optionally, treating the modified surface of the electrode after expiry of the predetermined period of time; and
optionally, examining or testing the modified surface to observe and/or characterize any change in properties thereof.

According to a further aspect, the present invention provides a method for grafting a monomeric or polymeric organic film onto an electrically conductive or semi-conductive surface. This method comprising: reacting a surface with a solution comprising at least one compound comprising one or more molecules having the formula (1), (2), (3), (4), (5), or (6), as described above.

In one embodiment of the invention, the method of the present invention further comprises an electrode and at least one counter electrode, and applying a potential between the electrode and the at least one counter electrode. Optionally, the method further comprises a reference electrode.

In one aspect of the present invention, the electrode is a carbon electrode. The at least one counter electrode comprises platinum, and the reference electrode comprises silver-silver chloride.

In yet another aspect of the invention, the electrically conductive surface is modified by applying at least one potential scan of 0 V to 0.9 V vs the reference electrode at a scan rate of 100 mV/s.

According to another aspect, the present invention further comprises the step of washing the surface.

According to yet another aspect, the present invention the surface is sonicated in a buffer solution.

In one embodiment of the invention, the electrically conductive or semi-conductive surface comprises at least one microparticle or at least one nanoparticle.

In one embodiment of the invention, the solution further comprises an oxidizing agent.

### DETAILED DESCRIPTION

Further aspects, features and advantages of this invention will become apparent from the detailed description which follows.

As noted above, in its broader aspects, the present invention is directed to one or more molecules having a structural formula of one or more structural formulas (1) to (10), as described above. The present invention is also directed to compounds comprising, consisting of, or consisting essentially of one or more molecules (1) to (10). Such compounds may optionally be in the form of a material or substrate having a surface modified by one or more molecules (1) to (10).

A modified surface of the present invention can be formed according to a number of alternative methods.

Methods performed according to the principles of the present invention may generally comprise one or more of the following steps, which may or may not be performed in the order below:
optionally, preparing the surface of the electrode to be modified by one or more molecules (1) to (10);
preparing a solution comprising one or more molecules (1) to (10);
placing the solution into communication with the surface of the electrode to be modified;
applying a predetermined potential between the surface of the electrode to be modified and the at least one counter electrode, for a predetermined period of time;
optionally, treating the modified surface of the electrode after expiry of the predetermined period of time; and
optionally, examining or testing the modified surface to observe and/or characterize any change in properties thereof.

In an exemplary embodiment, conductive or semi-conductive surfaces are modified in a voltaic cell. In this non-limiting embodiment, an electrode and at least one counter electrode are connected by an external circuit. Potential is applied between the electrode and the at least one counter electrode to obtain an electrode, wherein at least a portion of which forms the modified surface. Optionally, the potential is read by a voltmeter.

Electrodes, counter electrodes, and/or reference electrodes according to the invention can be materials known in the art, including, but not limited to, carbon, Pt, and or Ag/AgCl electrodes. Electrodes and counter electrodes of the present invention can be of the type obtainable from CH Instruments, Inc. of Austin, Tex.

Electrodes according to the present invention can be prepared by the following non-limiting example. A glassy carbon electrode is first polished with sand paper having 1500 grit and is ultrasonicated in deionized water (D.I. water) for about 2 minutes. The carbon electrode is then polished again with sand paper having 2500 grit, and ultrasonicated in D.I. water for about 2 minutes. The electrode is polished on a polishing cloth with alumina micro beads paste, and ultrasonicated again for about 2 minutes. Polishing cloths of the present invention can be of the type obtainable from Buehler, Ltd. of Lake Bluff, Ill.

The polished electrode can then optionally be electrochemically etched with an acid. Suitable acids include, but are not limited to, sulfuric acid, phosphoric acid, nitric acid, etc. In a non-limiting example, the electrode is etched with 1 M sulfuric acid at 1.8 V for about 5 minutes. The etched electrode is soaked in a base to etch the remaining alumina beads from the surface. Suitable bases include, but are not limited to, inorganic bases including alkali bases, alkaline bases, etc. In one non-limiting example, the electrode is soaked in 1 M potassium hydroxide for about 5 minutes. The electrode is treated with acid and at least one potential scan is applied until the surface exhibits minimal variances between potential scans on a voltammogram. In a non-limiting example, the electrode is treated with 1 M sulfuric acid at about −0.5 V to about 1.2 V at 100 mV/s for 25 cycles.

According to an illustrative example, a solution comprising one or more monomer and/or one or more polymer is prepared in a buffer. Buffers according to the invention can be acidic, basic, or neutral. A solution comprising one or more monomer and/or one or more polymer according to the invention can have a concentration in the range of about 0.1 mM to 20 mM, preferably 5 mM to 15 mM. In an exemplary embodiment, the solution comprising one or more monomer and/or one or more polymer is a 10 mM solution prepared in a phosphate buffer having a pH of 7.2 at room temperature.

The electrochemical properties of the surface are optionally tested in a buffer solution comprising a redox probe prior to surface modification. In an exemplary example, the redox probe is K₄Fe(CN)₆, and the buffer solution to test the surface comprises 10 mM K₄Fe(CN)₆ in a buffer having a pH of 7.2 at room temperature. In a non-limiting example, the current obtained before surface modification (i_{a,unmodified}) is measured by applying −0.1 V to 0.65 V at 25 mV/s for one cycle. The surface area and other electrode kinetic properties reflective of the unmodified surface can be estimated from the measured current i_{a,unmodified}.

In accordance with the voltammetry method, surface modification is performed by applying potential to the surface. In a non-limiting example, 0 V to 1 V is applied to the surface at 100 mV/s for ten cycles.

Optionally, the surface is washed after surface modification to remove the weakly adsorbed one or more monomer and/or one or more polymer. Suitable methods for washing the surface, include, but are not limited to, agitating the surface to remove the weakly adsorbed one or more monomer and/or one or more polymer. In an exemplary embodiment, the surface is first ultrasonicated in a buffer for about 1 minute to 20 minutes, preferably about 10 minutes, and then ultrasonicated in ethanol for about an additional 1 minute to 20 minutes, preferably about 10 minutes.

The electrochemcial properties of the surface are optionally tested in a buffer solution comprising a redox probe after surface modification. In an exemplary example, the redox probe is K₄Fe(CN)₆, and the buffer solution to test the surface comprises 10 mM K₄Fe(CN)₆ in a buffer having a pH of 7.2 at room temperature. In a non-limiting example, the current obtained after surface modification (i_{a,modified}) is measured by applying −0.1 V to 0.65 V at 25 mV/s for one cycle.

Optionally, the monomer or polymer blocking percentage is calculated from the current difference prior to surface modification and after surface modification according to the following equation:

blocking percentage=(i_{a,unmodified}−i_{a,modified})+i_{a,unmodified.}

When the polymer and or monomer molecules attach to the surface they form a dielectric layer. This layer does not allow the K₄Fe(CN)₆ closer to the electrode surface for electron transfer to occur. This reduces the anodic and cathodic current peaks in the voltammetric scan. The extent of surface attachment can be estimated using blocking % calculations. For example using the anodic peak current obtained before modification i_{a,unmodified} (elaborated in [00031]) and subtracting the anodic peak current obtained after modification i_{a,modified} (step [00034]) blocking %=(i_{a,unmodified}−i_{a,modified})/i_{a,unmodified} can be obtained. This blocking percentage provides the percentage of surface area unavailable for electron transfer reaction between K₄Fe(CN)₆ (redox probe) and the electrode. If the surface is blocked 100% then i_{a,modified}=0.

While elements of the invention have been described, it will be appreciated by those of ordinary skill in the art that modifications can be made to the structure and method of the invention without departing from the spirit and scope of the invention as a whole.

The composition s described herein are intended to encompass compositions, which consist of, consist essentially of, as well as comprise, the various constituents identified herein, unless explicitly indicated to the contrary.

Any numbers expressing quantities of ingredients, constituents, reaction conditions, and so forth used in the specification are to be interpreted as encompassing the exact numerical values identified herein, as well as being modified in all instances by the term “about.” Notwithstanding that the numerical ranges and parameters setting forth, the broad scope of the subject matter presented herein are approximations, the numerical values set forth are indicated as precisely as possible. Any numerical value, however, may inherently contain certain errors or inaccuracies as evident from the standard deviation found in their respective measurement techniques. None of the features recited herein should be interpreted as invoking 35 U.S.C. §112, paragraph 6, unless the term “means” is explicitly used.

What is claimed is:

1. A compound comprising one or more molecules, the molecules having a formula (1), (2), (3), (4), (5), or (6)
wherein: 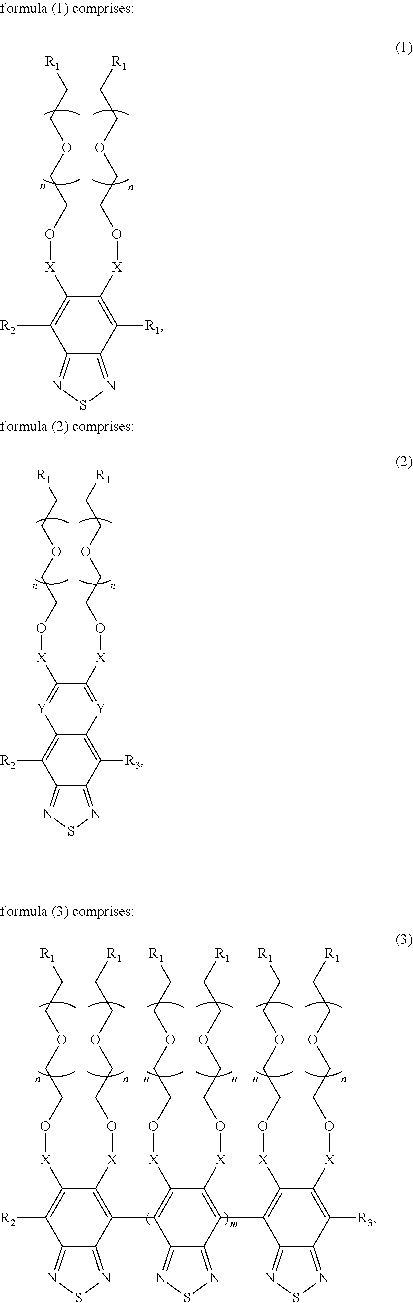 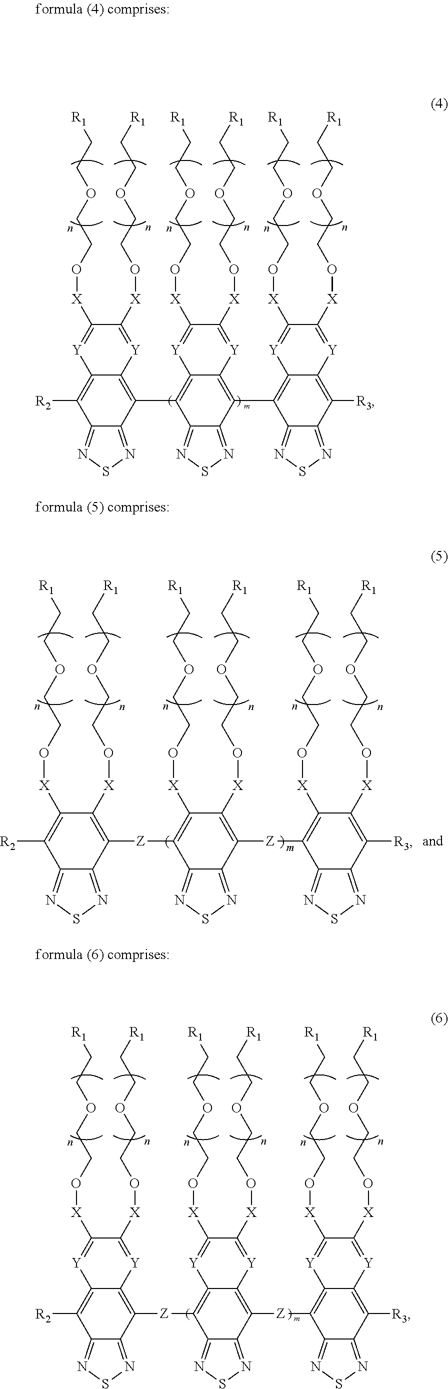
wherein
R₁=OH, COOH, NH₂, SO₃H, SR₄COOH, SR₄SO₃H, maleimide, N-hydroxysuccinimide, a protein, a nucleic acid, or a nanoparticle;
R₂=H, an alkene, an aromatic monomer, an aromatic oligomer, a heterocyclic monomer, or a heterocyclic oligimer;
R₃=R₂, a ligand, a metal, a protein, a nucleic acid, or a nanoparticle;
R₄=—(CH₂)ₜ—, —(CH₂—O—CH₂)ₜ—, an alkene, a polyalkene, an aromatic monomer, an aromatic polymer, a heterocyclic monomer, or a heterocyclic polymer;
X=N, S, O, P, —(CH₂)ₜ—, an aromatic monomer, or a heterocyclic monomer,
Y=N, S, O, or P;
Z=—(CH₂)ₜ—, an alkene, a polyalkene, an aromatic monomer, an aromatic polymer, a heterocyclic monomer, or a heterocyclic polymer;
m is 1 to 20;
n is 1 to 20; and
t is 1 to 20.

2. The compound of claim 1, wherein R₁ is SR₄COOH.

3. The compound of claim 2, wherein R₄ is a methylene bridge or a carbene.

4. The compound of claim 1, wherein R₂ is an aromatic monomer.

5. The compound of claim 4, wherein R₂ is benzothiadiazole, derivatives of benzothiadiazole, thiophene, or derivatives of thiophene.

6. The compound of claim 5, wherein R₂ is a thiophene.

7. The compound of claim 1, wherein R₂ is a metal.

8. The compound of claim 7, wherein R₂ is bromine.

9. The compound of claim 1, wherein formula (3) further comprises a molecule having structural formula (7), and
wherein formula (7) comprises:

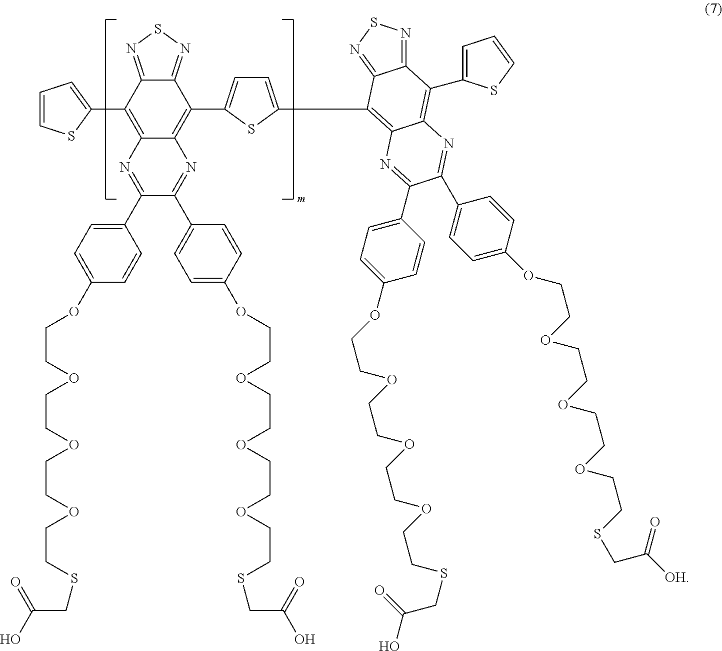

10. The compound of claim 1, wherein formula (3) further comprises a molecule having structural formula (8), and
wherein formula (8) comprises:

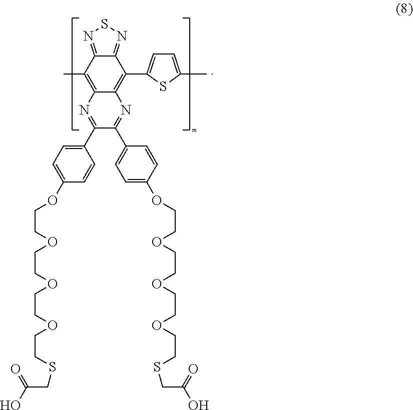

11. The compound of claim 1, wherein formula (1) further comprises a molecule having structural formula (9), and
wherein formula (9) comprises:

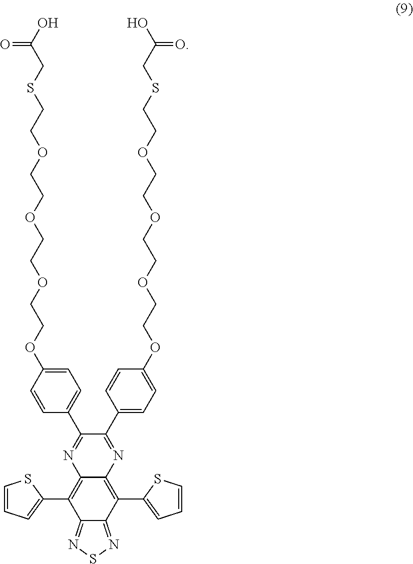 [SMILES: O=C(O)CSCCOCCOCCOCCOc1ccc(-c2nc3c(-c4cccs4)c4nsnc4c(-c4cccs4)c3nc2-c2ccc(OCCOCCOCCOCCSCC(=O)O)cc2)cc1]

12. The compound of claim 1, wherein formula (2) further comprises a molecule having structural formula (10), and
wherein formula (10) comprises:

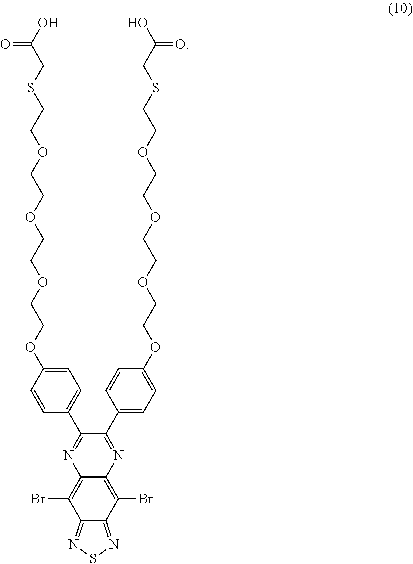 [SMILES: O=C(O)CSCCOCCOCCOCCOc1ccc(-c2nc3c(Br)c4nsnc4c(Br)c3nc2-c2ccc(OCCOCCOCCOCCSCC(=O)O)cc2)cc1]

13. A method for grafting a monomeric or polymeric organic film on an electrically conductive or semi-conductive surface, said method comprising:
reacting a surface with a solution comprising at least one compound comprising one or more molecules, the molecules having a formula (1), (2), (3), (4), (5), or (6), 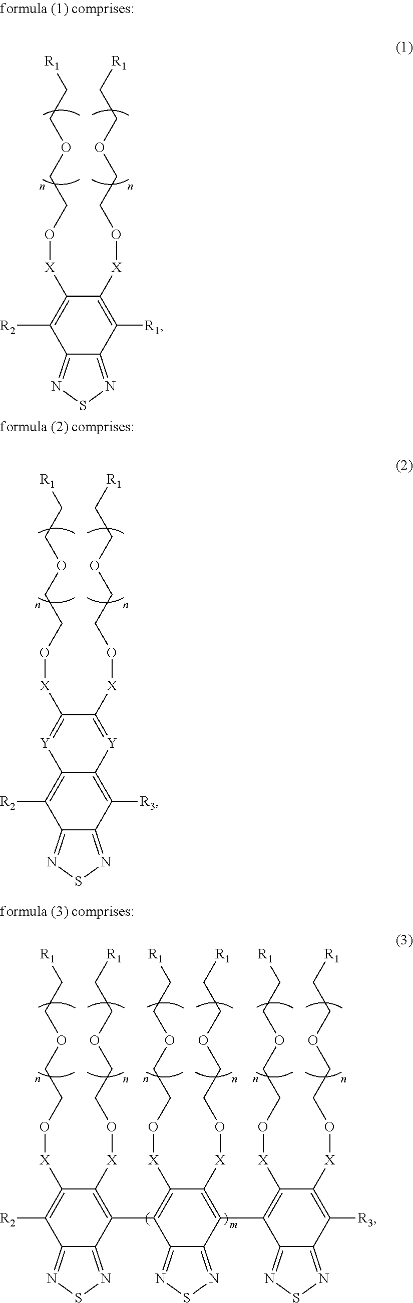 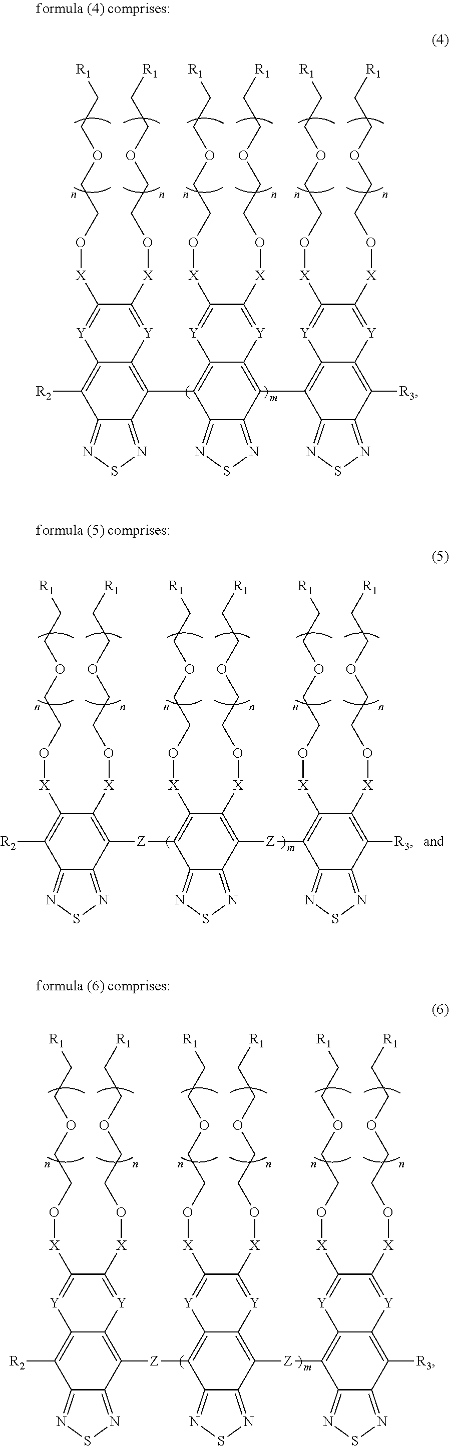
further wherein:
R₁=OH, COOH, NH₂, SO₃H, SR₄COOH, SR₄SO₃H, maleimide, N-hydroxuccinimide, a protein, a nucleic acid, or a nanoparticle;
R₂=H, an alkene, an aromatic monomer, an aromatic oligomer, a heterocyclic monomer, or a heterocyclic oligomer;
R₃=R₂, a ligand, a metal, a protein, a nucleic acid, or a nanoparticle;
R₄=—(CH₂)ₜ—, —(CH₂—O—CH₂)ₜ—, an alkene, a polyalkene, an aromatic monomer, an aromatic polymer, a heterocyclic monomer, or a heterocyclic polymer;
X=N, S, O, or P or —(CH₂)ₜ—, an aromatic monomer, a heterocyclic monomer;
Y=N, S, O, or P;
Z=—(CH₂)ₜ—, an alkene, a polyalkene, an aromatic monomer, an aromatic polymer, a heterocyclic monomer, or a heterocyclic polymer;
m is 1 to 20;
n is 1 to 20; and
t is 1 to 20.

14. The method of claim 13, further comprising an electrode and at least one counter electrode, wherein at least a portion of the electrode forms the surface, and applying a potential between the electrode and the at least one counter electrode.

15. The method of claim 14, further comprising a reference electrode.

16. The method of claim 14, wherein the electrode is a carbon electrode.

17. The method of claim 14, wherein the at least one counter electrode comprises platinum.

18. The method of claim 15, wherein the reference electrode comprises silver.

19. The method of claim 18, wherein the reference electrode further comprises AgCl.

20. The method of claim 15, wherein the surface modification comprises applying 0 V to 0.9 V of current vs the reference electrode at a scan rate of 100 mV/s to the surface.

21. The method of claim 13, further comprising washing the surface.

22. The method of claim 13, further comprising sonicating the surface.

23. The method of claim 22, wherein the sonicating is conducted in a buffer.

24. The method of claim 13, wherein the surface comprises at least one microparticle.

25. The method of claim 13, wherein the surface comprises at least one nanoparticle.

26. The method of claim 13, wherein the solution comprises an oxidizing agent.
